SEQUENCE LISTING (4 sequences)

SEQ ID NO: 1 - DNA, 1398 bp, synthetic construct
  source 1..1398: mol_type = other DNA
ctcccgaaggttaagctacctacttcttttgcaacccactcccatggtgtgacgggcggtgtgtacaaggcccgggaacgtattcaccgtggcattctgatccacgattactagcgattccgacttcatggagtcgagttgcagactccaatccggactacgacgcactttatgaggtccgcttactctcgcgaggtcgcttctctttgtatgcgccattgtagcacgtgtgtagccctggtcgtaagggccatgatgacttgacgtcatccccaccttcctccagtttatcactggcagtctcctttgagttcccggccggaccgctggcaacaaaggataagggttgcgctcgttgcgggacttaacccaacatttcacaacacgagctgacgacagccatgcagcacctgtctcacagttcccgaaggcaccaatccatctctggaaagttctgtggatgtcaagaccaggtaaggttcttcgcgttgcatcgaattaaaccacatgctccaccgcttgtgcggscccccgtcaattcatttgagttttaaccttgcggcccgtactcccccaggcggtctatttaacgcgttagctccggaagccacgcctcaagggcacaacctccaaatagacatcgtttacggcgtggactaccagggtatctaatcctgtttgctccccacgctttcgcacctgagcgtcagtcttcgtccaggaggccgccttcgccaccggtattcctccagatctctacgcatttcaccgctacacctggaattctacctccctctacgagactcaagcctgccagtttcggatgcagttcccaggttgagcccggggatttcacatccgacttgacagaccgcctgcgtgcgctttacgcccagtaattccgattaacgcttgcaccctccgtattaccgcggctgctggcacggagttagccggtgcttcttctgcgggtaacgtcaatgagcaaggttattaaccttactcccttcctccccgctgaaagtgctttacaacccgaaggccttcttcacacacgcggcatggctgcatcaggcttgcgcccattgtgcaatattccccactgctgcctcccgtaggagtctggaccgtgtctcagttccagtgtggctggtcatcctctcagaccagctagggatcgtcgcctaggtgagccgttaccccacctactagctaatcccatctgggcacatctgatggcaagaggcccgaaggtccccctctttggtcttgcgacgttatgcggtattagctaccgtttccagtagttatccccctccatcaggcagtttcccagacattactcacccgtccgccactcgtcacccgagagcaagctctctgtgctaccgttcgact

SEQ ID NO: 2 - DNA, 25 bp, synthetic construct
  source 1..25: mol_type = other DNA
tcatcaataacaatccctgcgacgc

SEQ ID NO: 3 - DNA, 25 bp, synthetic construct
  source 1..25: mol_type = other DNA
agccttcgcaccgcaccggaataac

SEQ ID NO: 4 - DNA, 416 bp, synthetic construct
  source 1..416: mol_type = other DNA
aacaggatttagataccctggtagtccacgctgtaaacgatgtcgatttggaggttgtgcccttgaggcgtggcttccggagctaacgcgttaaatcgaccgcctggggagtacggccgcaaggttaaaactcaaatgaattgacgggggcccgcacaagcggtggagcatgtggtttaattcgatgcaacgcgaagaaccttacctggtcttgacatccacagaacttagcagagatgctttggtgccttcgggaactgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtgggggatgacgt

(19) United States
(12) Patent Application Publication
(10) Pub. No.: US 2026/0174084 A1
(43) Pub. Date: Jun. 25, 2026

(54) NITROGEN-FIXING BACTERIA AND USE THEREOF

(71) Applicant: BEIJING LIFEWE BIOTECHNOLOGY INSTITUTE CO., LTD., Beijing (CN)

(72) Inventors: Jilong WANG, Beijing (CN); Zhiqiang ZHOU, Beijing (CN); Chengwei MA, Beijing (CN); Xiang JIN, Beijing (CN)

(21) Appl. No.: 18/833,256

(22) PCT Filed: Apr. 17, 2024
(86) PCT No.: PCT/CN2024/088368

§ 371 Date: Jul. 25, 2024

(30) Foreign Application Priority Data
Sep. 28, 2023 (CN) 202311268005.8
Oct. 7, 2023 (CN) 202311277829.1

(51) Int. Cl. A01N 63/20; A01P 21/00; C12N 1/205; C12R 1/01; C12R 1/22

(52) U.S. Cl. CPC A01N 63/20; A01P 21/00; C12N 1/205; C12R 2001/01; C12R 2001/22

(57) ABSTRACT

The present invention relates to the technical field of microorganisms, and in particular to nitrogen-fixing bacteria and use thereof. The nitrogen-fixing bacteria include Kosakonia sacchari LWNF014 and/or Klebsiella variicola LWNF004. The Kosakonia sacchari LWNF014 was screened from the farmland in Gongzhuling, Jilin Province, with an accession number of CGMCC No. 28352, and the Klebsiella variicola LWNF004 was screened from the farmland in Gongzhuling, Jilin Province, with an accession number of CGMCC No. 28351. The aforementioned strains have nitrogen-fixing capability, can form an efficient interaction with corn, and can accelerate the growth process of corn, improving the chlorophyll content of leaves. The related strains are used for preparing an efficient microbial agent, which has the advantages of low production cost, simple operations, no secondary pollution to the soil, and the like.

### CROSS-REFERENCE

The present application is based on the Chinese patent application with application Ser. No. 202311268005.8 filed on Sep. 28, 2023, and the Chinese patent application with application No. 202311277829.1 filed on Oct. 7, 2023, and claims the priority of the aforementioned Chinese patent applications, the contents of which are incorporated herein by reference in their entireties.

### TECHNICAL FIELD

The present invention relates to the technical field of microorganisms, and in particular to nitrogen-fixing bacteria and use thereof.

### BACKGROUND

Soil microorganisms include: bacteria, fungi, actinomycetes, and the like, which are large in number and wide in variety, and play different functions in the soil. Some of the microorganisms can induce plant diseases, namely phytopathogenic microorganisms; in addition, there are also some microorganisms that can promote plant growth, namely plant growth-promoting microorganisms. In recent years, as the problems of the global environment and the world climate have become increasingly severe, the concern and demand of various countries for the green, environmentally friendly, and low-carbon industry are increasing, and such microorganisms for promoting plant growth are gradually valued and favored in modern agriculture. As a new type of fertilizer for improving crop growth, the sustainability and the soil-friendly property of such microorganisms are incomparable to those of traditional chemical fertilizers. In addition, the microbial fertilizer also has the significant advantages of a mild production process, low energy consumption, little pollution, and the like, and has great advantages and potential in the field of fertilizers. Plant growth-promoting bacteria have different growth-promoting mechanisms of action on crops. Some of the plant growth-promoting bacteria have the effects of nitrogen fixation, phosphorus solubilization, and the like, and can provide elements such as nitrogen and phosphorus for plants; some of the plant growth-promoting bacteria can inhibit the growth of phytopathogenic microorganisms and protect crops from being damaged by soil-borne diseases, and regardless of the growth-promoting mechanism, the colonization capability of the plant growth-promoting bacteria in the plants directly affects the growth-promoting effect. The growth-promoting bacteria with strong colonization capability and relatively long duration of colonization generally have a better growth-promoting effect.

The nitrogen element is one of the three essential nutrient elements in the process of plant growth and development, and is an important component of proteins, nucleic acids and chlorophyll in plants. Therefore, it is an essential component of enzymes in plants. Simultaneously, nitrogen is also a component of some vitamins and alkaloids in plants. A large number of research results have proved that the amount of the nitrogen fertilizer applied directly affects the growth of crops and the crop yields, and can also significantly affect the physiological metabolism of the crops during growth. In order to achieve the purpose of yield increase, traditional fertilization generally uses chemical fertilizers in excess, which has, is, and will continue to cause a series of negative problems, such as soil hardening, changes in physicochemical properties, massive deaths of beneficial bacteria, earthworms, and the like in the soil, excessive enrichment of certain elements, and the like. In the short term, the direct effect of these negative problems is a decrease in crop yields and an increase in environmental pollution. In the long term, excessive use of chemical fertilizers reduces the self-regeneration and recycling capability of soil fertility, and further, more chemical fertilizers are needed, thereby forming a vicious circle. Accordingly, it is of great significance to explore other green pollution-free ways to replace the current fertilizer application through microbial technology to meet the sustainable development of agriculture and the like. Nitrogen-fixing bacteria exist naturally in nature, particularly in the soil, which can convert elemental nitrogen in the air into ammonium ions that can be used by plants through their own nitrogen-fixing function, and thus provide nitrogen fertilizer nutrition for the plants. People try to separate efficient nitrogen-fixing strains from soil microbial communities, which can be symbiotic or association with crops and partially or completely replace industrial nitrogen fertilizers to support crop growth. However, the known nitrogen-fixing bacteria mostly have the problems of weak nitrogen-fixing capability, insignificant plant growth-promoting effects, and the like, and thus, they are not suitable as supplements or substitutes for industrial nitrogen fertilizers.

In conclusion, a primary nitrogen-fixing strain that has the plant growth-promoting effect is screened from the soil and used as a microbial fertilizer to partially or completely replace the chemical fertilizer so as to improve the growth and development of the plants, the ecological environment of the soil is not damaged or even improved while achieving stable and increased yields of the plants, and thus, the nitrogen-fixing strain has great demand potential.

### SUMMARY

An objective of the present invention is to provide a strain with a nitrogen-fixing function and related use thereof, wherein the strain comprises Kosakonia sacchari LWNF014 and/or Klebsiella variicola LWNF004.

Another objective of the present invention is to provide a strain with a plant growth-promoting effect, wherein the strain comprises Kosakonia sacchari LWNF014 and/or Klebsiella variicola LWNF004.

Preferably, the strain can stably colonize a corn root for more than 2 months.

Another objective of the present invention is to provide Kosakonia sacchari LWNF014 and/or Klebsiella variicola LWNF004 with one or more of the aforementioned capabilities simultaneously, and related use thereof.

In order to achieve the aforementioned objectives, the present invention provides the following technical solutions:

A strain of Kosakonia sacchari LWNF014 has been deposited at the China General Microbiological Culture Collection Center (abbreviated as CGMCC, address: No. 3, Yard No. 1, West Beichen Road, Chaoyang District, Beijing, Institute of Microbiology, Chinese Academy of Sciences) on Sep. 4, 2023, with an accession number of CGMCC No. 28352, and is classified and named as Kosakonia sacchari.

The Kosakonia sacchari LWNF014 of the present invention is obtained by multiple screening from the soil around the roots and rhizosphere of corn plants in Gongzhuling, Jilin Province, can grow on a nitrogen-free medium, and has nitrogen-fixing capability. The 16S rRNA coding gene sequencing is performed on the strain, and the sequence of the strain is set forth in SEQ ID NO. 1. Based on the aforementioned results of various physiological and biochemical tests, the strain is identified as belonging to the genus Kosakonia at a molecular level, and is classified and named as Kosakonia sacchari. The Kosakonia sacchari LWNF014 is proven to have effects of increasing the chlorophyll content of corn, increasing the nitrogen content of leaves, and increasing the biomass of corn simultaneously by a corn seedlings experiment.

A strain of Klebsiella variicola LWNF004 has been deposited at the China General Microbiological Culture Collection Center (address: No. 3, Yard No. 1, West Beichen Road, Chaoyang District, Beijing, Institute of Microbiology, Chinese Academy of Sciences) on Sep. 4, 2023, and is classified and named as Klebsiella variicola with an accession number of CGMCC No. 28351.

The Klebsiella variicola LWNF004 of the present invention is obtained by multiple screening from the soil around the roots and rhizosphere of corn plants in Gongzhuling, Jilin Province, and can grow on a nitrogen-free medium, and acetylene reduction experiment results show that the strain has nitrogen-fixing capability. The 16S rRNA coding gene sequencing is performed on the strain, and the sequence of the strain is set forth in SEQ ID NO. 4. Based on the aforementioned results of various physiological and biochemical tests, the strain is identified to be Klebsiella variicola at a molecular level.

The Klebsiella variicola LWNF004 is proven to be capable of stably colonizing a corn root for more than 2 months and to have the effect of promoting the growth of the corn root by a corn seedlings experiment.

The present invention provides a strain of Kosakonia sacchari LWNF014, wherein the accession number of the Kosakonia sacchari LWNF014 is CGMCC No. 28352.

The present invention provides a strain of Klebsiella variicola LWNF004, wherein the accession number of the Klebsiella variicola LWNF004 is CGMCC No. 28351.

The present invention provides use of a strain in promoting plant growth, wherein the strain comprises the aforementioned Kosakonia sacchari LWNF014 and/or the aforementioned Klebsiella variicola LWNF004.

Preferably, the plant can be a crop including a food crop and a cash crop; optionally, the cash crop includes an oil crop and a vegetable crop; the food crop includes a grain crop, a tuber crop, and an edible legume crop; further preferably, the crop is a vegetable crop including corn, beans, cucumbers or peppers, more preferably corn.

Preferably, the Kosakonia sacchari LWNF014 and/or the Klebsiella variicola LWNF004 can improve nitrogen fixation, promote photosynthesis, and increase the chlorophyll content, or promote a growth-promoting effect for plant growth.

Preferably, the promotion of plant growth includes a promotion of plant root growth or a promotion of plant aerial part growth.

Preferably, the Kosakonia sacchari LWNF014 can improve nitrogen fixation, promote photosynthesis, and increase the chlorophyll content.

Preferably, the Klebsiella variicola LWNF004 stably colonizes plant roots for more than 2 months, providing nitrogen source nutrition for the plants and promoting a clustering effect of plant root growth.

The present invention provides use of the aforementioned Kosakonia sacchari LWNF014 and/or the aforementioned Klebsiella variicola LWNF004 as nitrogen-fixing bacteria.

The present invention further provides a microbial nitrogen-fixing agent, wherein the microbial agent comprises the aforementioned Kosakonia sacchari LWNF014 and/or the aforementioned Klebsiella variicola LWNF004.

Preferably, the microbial nitrogen-fixing agent can be applied to a plant as a fertilizer, and the plant is preferably a crop.

Preferably, the crop includes a food crop and a cash crop; optionally, the cash crop includes an oil crop and a vegetable crop; the food crop includes a grain crop, a tuber crop, and an edible legume crop; further preferably, the crop is a vegetable crop including corn, beans, cucumbers or peppers, more preferably corn.

In the aforementioned microbial agent, an effective viable count of the Kosakonia sacchari LWNF014 is preferably 10¹⁰-10¹¹ CFU/g.

In the aforementioned microbial agent, an effective viable count of the Klebsiella variicola LWNF004 is preferably 10¹⁰-10¹¹ CFU/g.

The aforementioned microbial agent can be prepared by adopting conventional microbial agent preparation methods in the art, and the microbial agent can be a microbial agent in various forms, such as a liquid microbial agent or a solid microbial agent, preferably a solid microbial agent.

The solid microbial agent is preferably prepared by adopting a fermentation and lyophilization method.

The present invention further provides a plant growth-promoting microbial agent, wherein the microbial agent comprises the aforementioned Kosakonia sacchari LWNF014 and/or the aforementioned Klebsiella variicola LWNF004.

Preferably, the microbial growth-promoting agent can be applied to a plant as a fertilizer, and the plant is preferably a crop.

Preferably, the crop includes a food crop and a cash crop; optionally, the cash crop includes an oil crop and a vegetable crop; the food crop includes a grain crop, a tuber crop, and an edible legume crop; further preferably, the crop is a vegetable crop including corn, beans, cucumbers or peppers, more preferably corn.

In the aforementioned microbial agent, an effective viable count of the Kosakonia sacchari LWNF014 is preferably 10¹⁰-10¹¹ CFU/g.

In the aforementioned microbial agent, an effective viable count of the Klebsiella variicola LWNF004 is preferably 10¹⁰-10¹¹ CFU/g.

The aforementioned microbial agent can be prepared by adopting conventional microbial agent preparation methods in the art, and the microbial agent can be a microbial agent in various forms, such as a liquid microbial agent or a solid microbial agent, preferably a solid microbial agent.

The solid microbial agent is preferably prepared by adopting a fermentation and lyophilization method.

The present invention provides a method for preparing a microbial agent, wherein the aforementioned Kosakonia sacchari LWNF014 and/or the aforementioned Klebsiella variicola LWNF004 are activated to prepare a seed solution, which is subsequently inoculated into a fermentation medium for expansion to a stationary phase, and the microbial agent is obtained after separation.

The present invention provides a method for preparing a lyophilized powder microbial fertilizer, wherein a fermentation broth obtained by fermentation of the aforementioned Kosakonia sacchari LWNF014 and/or the aforementioned Klebsiella variicola LWNF004 is centrifuged and separated to obtain a bacterial precipitate, a lyoprotectant is subsequently added, and the mixture is lyophilized under an aseptic condition to prepare a lyophilized powder.

Preferably, the lyoprotectant is prepared by mixing skim milk powder, sodium glutamate, glycerol, sucrose, and water in a mass ratio of 75:15:70:10:330.

An effective viable count of the Kosakonia sacchari LWNF014 and/or Klebsiella variicola LWNF004 lyophilized powder is 10¹⁰-10¹¹ CFU/g, and the lyophilized powder can be stored for 6 months at room temperature without reduction of viability. The present invention provides a plant photosynthesis improver, wherein the plant photosynthesis improver comprises the aforementioned Kosakonia sacchari LWNF014 and/or the aforementioned Klebsiella variicola LWNF004, or the aforementioned microbial agent, the aforementioned plant growth-promoting microbial agent, the aforementioned microbial nitrogen-fixing agent, or the microbial agent prepared by the aforementioned method.

The present invention provides a plant nitrogen nutrition improver, wherein the plant nitrogen nutrition improver comprises the aforementioned Kosakonia sacchari LWNF014 and/or the aforementioned Klebsiella variicola LWNF004, or the aforementioned microbial agent, the aforementioned plant growth-promoting microbial agent, the aforementioned microbial nitrogen-fixing agent, or the microbial agent prepared by the aforementioned method.

The present invention provides a plant growth and development promoter, wherein the plant growth and development promoter comprises the aforementioned Kosakonia sacchari LWNF014 and/or the aforementioned Klebsiella variicola LWNF004, or the aforementioned microbial agent, the aforementioned plant growth-promoting microbial agent, the aforementioned microbial nitrogen-fixing agent, or the microbial agent prepared by the aforementioned method.

### Strain Deposition Information:

### 1) Strain LWNF014

Depositary institution: the China General Microbiological Culture Collection Center (CGMCC);
Address of the depositary institution: No. 3, Yard No. 1, West Beichen Road, Chaoyang District, Beijing;
Accession number: CGMCC No. 28352;
Deposition date: Sep. 4, 2023;
Classification and naming: Kosakonia sacchari.

### 2) LWNF004

Depositary institution: the China General Microbiological Culture Collection Center (CGMCC);
Address of the depositary institution: No. 3, Yard No. 1, West Beichen Road, Chaoyang District, Beijing;
Accession number: CGMCC No. 28351;
Deposition date: Sep. 4, 2023;
Classification and naming: Klebsiella variicola.

### BRIEF DESCRIPTION OF THE DRAWINGS

FIG. 1 is an electrophoretogram of nifH gene on Kosakonia sacchari LWNF014 genome by colony PCR assay.

FIG. 2 shows the effect of Kosakonia sacchari LWNF014 on the growth of corn seedlings after 7 days of treatment.

FIG. 3 shows the effect of Kosakonia sacchari LWNF014 on the growth of corn seedlings after 14 days of treatment.

FIG. 4 shows the effects of Kosakonia sacchari LWNF014 on chlorophyll and nitrogen content of corn seedlings after 7 days of treatment.

FIG. 5 shows the effects of Kosakonia sacchari LWNF014 on chlorophyll and nitrogen content of corn seedlings after 14 days of treatment.

FIG. 6 is a schematic diagram of the preparation process of a microbial agent.

FIG. 7 shows the effect of Klebsiella variicola LWNF004 on the fresh weight of corn roots.

FIG. 8 is a diagram showing a corn root sample in an abundance determination experiment of Klebsiella variicola LWNF004 in corn roots.

FIG. 9 is a diagram showing fluorescent chromogenic plates in the abundance determination experiment of Klebsiella variicola LWNF004 in corn roots.

### DETAILED DESCRIPTION

The present invention will be further illustrated with reference to the following specific examples. Unless otherwise specifically specified, the experimental conditions are all in accordance with conventional conditions well known to those skilled in the art.

### Example 1. Screening and Use of Nitrogen-Fixing Kosakonia sacchari LWNF014

1. Screening of Nitrogen-Fixing Kosakonia sacchari LWNF014

Soil and corn root samples were collected from the cornfield in Gongzhuling, Jilin Province, transported to the laboratory at low temperature. Nitrogen-fixing bacteria were isolated from the samples by grown on nitrogen-free medium KPM (the medium formula is shown in Table 1) plates. Monoclonal strains were purified by resteaking them on a nitrogen-free medium KPM plate. The coding gene of 16s rRNA was amplified by PCR and sequenced. After sequence alignment, one of the strains, LWNF014, was identified as Kosakonia sacchari belonging to the genus Kosakonia, and the sequence of the coding gene of 16s rRNA of the strain is set forth in SEQ ID NO. 1.

TABLE 1
KPM nitrogen-free medium formula

| Component | Content | |
|---|---|---|
| Na₂HPO₄ | 10.4 | g/L |
| KH₂PO₄ | 3.4 | g/L |
| CaCl₂•2H₂O | 26 | mg/L |
| MgSO₄ | 30 | mg/L |
| MnSO₄ | 0.3 | mg/L |
| Ferric citrate | 36 | mg/L |

| | |
|---|---|
| Sucrose | 1% (w/v) |

2. Identification of Nitrogen-Fixing Gene of Nitrogen-Fixing Strain Kosakonia sacchari LWNF014

The nitrogen-fixing gene nifH of Kosakonia sacchari LWNF014 was amplified by using primer pairs, Ks nifH-F: tcatcaataacaatccctgcgacgc (SEQ ID NO. 2) and KsnifH-R: agccttcgcaccgcaccggaataac (SEQ ID NO. 3), and the electrophoresis results (FIG. 1) of the PCR products show a specific band of about 2037 bp, which is consistent with the size of nifH in Klebsiella variicola, demonstrating that the strain carries the nitrogen-fixing gene nifH (FIG. 1).

3. Determination of Nitrogenase Activity in Nitrogen-Fixing Strain Kosakonia sacchari LWNF014

The strain in the logarithmic growth phase was transferred to an LB liquid medium and grown in a flask on a shaker at 220 RPM at 30° C. overnight. After the determination of OD₆₀₀ of the strain with a spectrophotometer, the bacteria were collected by centrifugation at 5500 RPM for 5 mint, washed 3 times with a nitrogen-free KPM liquid medium, suspended in a nitrogen-free KPM medium, and adjusted to OD600=0.2. 2 mL of the bacterial solution was added into each anaerobic tube, and an anaerobic tube containing 2 mL of a nitrogen-free KPM was used as a control (without addition of bacteria). The anaerobic tubes were sealed with stoppers to form a sealed state, and 2 mL of acetylene gas was injected into the anaerobic tubes. The anaerobic tubes were placed in an incubator at 30° C. for static culture for 16 h after being shaken evenly. 1 mL of gas was extracted from each anaerobic tube by a microsyringe and injected into a gas chromatograph to determine the ethylene content.

TABLE 2
Amount of product ethylene produced in determination
experiment of nitrogenase activity

| Treatment of enzyme | Peak area of product |
|---|---|
| activity experiment | ethylene (AU min) |
| Without addition of bacteria | ND |
| With addition of bacteria | 110082 ± 16430 |

As can be seen from the determination results in Table 2, when the Kosakonia sacchari LWNF014 was added into the anaerobic tube, it can catalyze the conversion of acetylene to ethylene, while no ethylene production was detected in the control check without the addition of bacteria. Based on the actual measured concentration/area ratio of acetylene standard gas, the nitrogenase activity of the Kosakonia sacchari LWNF014 can be calculated to be 0.35 nmol C₂H₄/h vial, which fully demonstrates that the Kosakonia sacchari LWNF014 has relatively high nitrogenase activity.

4. Effect of Kosakonia sacchari LWNF014 on Corn Growth

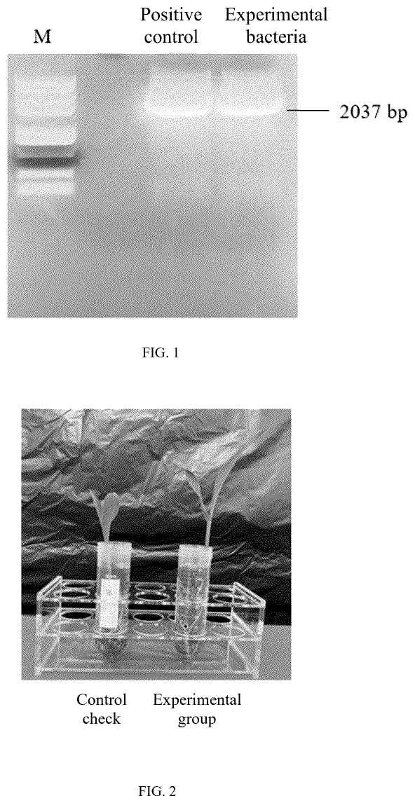

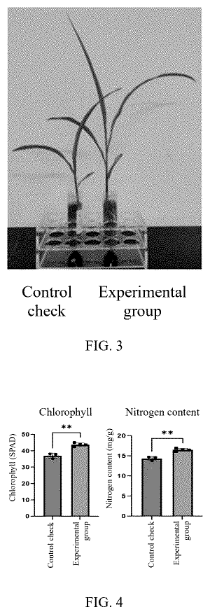

The Kosakonia sacchari LWNF014 was inoculated into an LB medium and cultured at 30° C. and 220 RPM for 16. The mixture was centrifuged at 5500 rpm for 5 mint to collect the bacteria, and the supernatant was discarded. The concentration of the bacteria was adjusted to 10⁹ CFU/mL with sterile PBS. Corn seeds were planted in 50 mL centrifuge tubes. In an experimental group, 200 μL of the prepared bacterial suspension was added, and subsequently, 10 mL of sterile water was added (right centrifuge tubes in FIG. 2 and FIG. 3). In a control check, 200 μL of sterile PBS and 10 mL of sterile water were added (left centrifuge tubes in FIG. 2 and FIG. 3). Each of the two treatments was repeated 4 times. After that, 5 mL of water was added every other day. The chlorophyll content (known as SPAD value) of the corn was determined by a chlorophyll meter after 3 weeks of planting to characterize the photosynthesis capability of the corn. The nitrogen content of the leaves can be calculated because the SPAD value and the nitrogen content of the plants have a specific proportional relation.

The corn growth is shown in FIG. 2, FIG. 3 and Table 3. When the corn was cultivated for 7 days, the height of the corn seedlings of the experimental group applied with the Kosakonia sacchari LWNF014 bacterial suspension was 20 cm, and the height of the corn seedlings of the control check not applied with the bacterial solution was 15 cm. The height of the corn seedlings of the experimental group was 33% higher than that of the control check, and the diameter of the corn seedlings of the experimental group was significantly thicker than that of the control check. When the corn was cultivated for 14 days, the corn seedlings of the experimental group entered the 4-leaf stage, and the corn seedlings of the control check were still in the 3-leaf stage. Moreover, the advantages in height and diameter of the corn seedlings of the experimental group over those of the control check continued to increase. The height of the corn seedlings of the experimental group was 139% of that of the control check, and the difference between the two was further increased compared with the 7-day stage. The aforementioned results fully demonstrate that the Kosakonia sacchari LWNF014 can accelerate corn seedling growth, make the corn seedlings grow taller and thicker, and shorten the growth cycle of the corn.

TABLE 3
Statistics of growth height and diameter of corn seedlings

| | | Height of | Diameter of |
|---|---|---|---|
| Cultivation time | Treatment | corn seedling | corn seedling |
| 7 days | Experimental group | 20 cm | thick |
| | Control check | 15 cm | thinner |
| 14 days | Experimental group | 53 cm | thick |
| | Control check | 38 cm | thinner |

As can be seen from the results in FIG. 4 and FIG. 5, when the corn was cultivated for 7 days, the average chlorophyll content of the corn seedlings in the control check not applied with the bacterial solution was 37 SPAD and the average nitrogen content of the leaves was 14 mg/g. The chlorophyll content of the corn seedlings in the experimental group applied with the Kosakonia sacchari LWNF014 bacterial suspension was 43.8 SPAD and the nitrogen content of the leaves was 16 mg/g, which were improved by 18% and 14%, respectively, compared with the control check. When the corn was cultivated for 14 days, the chlorophyll content of the corn seedlings in the control check not applied with the bacterial solution was 29 SPAD and the nitrogen content of the leaves was 12 mg/g. The chlorophyll content of the corn seedlings in the experimental group applied with the Kosakonia sacchari LWNF014 bacterial suspension was 38.5 SPAD and the nitrogen content of the leaves was 14 mg/g, which were improved by 32% and 16%, respectively, compared with the control check. The aforementioned results show that the application of Kosakonia sacchari LWNF014 can increase the synthesis of chlorophyll and the accumulation of nitrogen-containing substances in the corn seedlings and promote the growth of corn seedlings.

### 5. Preparation of Microbial Fertilizer

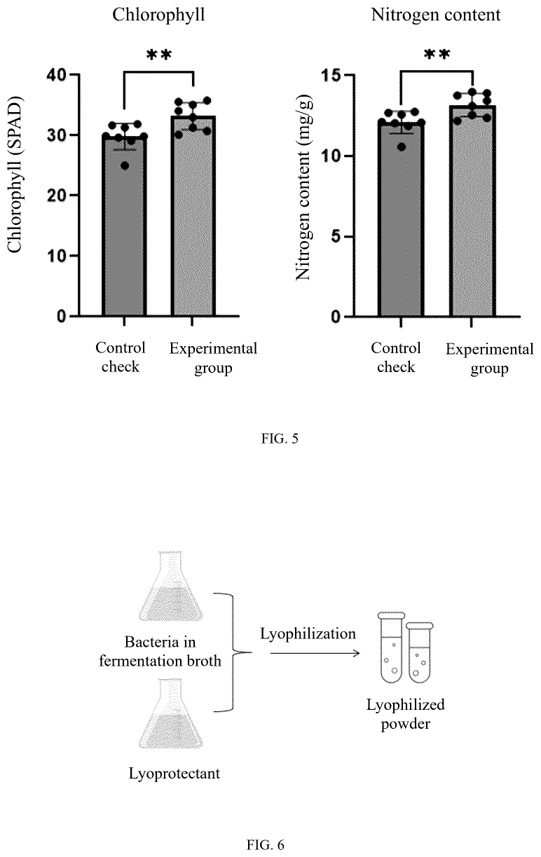

In order to facilitate storage and transportation, the Kosakonia sacchari LWNF014 screened in step 2 was prepared into a lyophilized powder microbial fertilizer. The specific procedures were as follows (the preparation process is shown in FIG. 6):
(1) a fermentation broth of the Kosakonia sacchari LWNF014 was centrifuged and separated to obtain a bacterial precipitate;
(2) a lyoprotectant was prepared by mixing skim milk powder, sodium glutamate, glycerol, sucrose, and water in a mass ratio of 75:15:70:10:330;
(3) the lyoprotectant was added to the bacterial precipitate obtained in the aforementioned step (1) in a mass ratio of 1:1, the bacteria and the protectant were mixed fully and equilibrated for 60 min, and subsequently, the mixture was frozen at −20° C. overnight; and
(4) the mixture was lyophilized under an aseptic condition to prepare a lyophilized powder for later use.

An effective viable count of the Kosakonia sacchari LWNF014 lyophilized powder is 10¹⁰-10¹¹ CFU/g, and the lyophilized powder can be stored for 6 months at room temperature without reduction of viability.

### Example 2. Screening and Use of Nitrogen-Fixing Klebsiella variicola LWNF004

1. Screening of Nitrogen-Fixing Klebsiella variicola LWNF004

Soil and corn root samples were collected from the farmland in Gongzhuling, Jilin Province, transported to the laboratory at low temperature, and plated on nitrogen-free medium KPM (the medium formula is shown in Table 4) plates for culture. Monoclonal strains were picked from the plate, and resteaked on a nitrogen-free medium KPM plate for purification. The coding gene of 16s rRNA was amplified by PCR and sequenced. After sequence alignment, one of the strains, LWNF004, was screened and identified as Klebsiella variicola, and the sequence of the coding gene of 16s rRNA of the strain is set forth in SEQ ID NO. 4.

TABLE 4
KPM nitrogen-free medium formula

| Component | Content | |
|---|---|---|
| Na₂HPO₄ | 10.4 | g/L |
| KH₂PO₄ | 3.4 | g/L |
| CaCl₂•2H₂O | 26 | mg/L |
| MgSO₄ | 30 | mg/L |
| MnSO₄ | 0.3 | mg/L |
| Ferric citrate | 36 | mg/L |

| | |
|---|---|
| Sucrose | 1% (w/v) |

2. Identification of Nitrogen-Fixing Capability of Klebsiella variicola LWNF004

The acetylene reduction method commonly used in the art was adopted to detect the nitrogenase activity of the Klebsiella variicola LWNF004.

The strain in the logarithmic growth phase was transferred to an LB liquid medium and grown in a flask on a shaker at 220 RPM at 30° C. overnight. After the determination of OD₆₀₀ of the strain with a spectrophotometer, the bacteria were collected by centrifugation at 5500 RPM for 5 mint, washed 3 times with a nitrogen-free KPM liquid medium, suspended in a nitrogen-free KPM medium, and adjusted to OD600=0.2. 2 mL of the bacterial solution was added into each anaerobic tube, and an anaerobic tube containing 2 mL of a nitrogen-free KPM was used as a control (without addition of bacteria). The anaerobic tubes were sealed with stoppers to form a sealed state, and 2 mL of acetylene gas was injected into the anaerobic tubes. The anaerobic tubes were placed in an incubator at 30° C. for static culture for 16 h after being shaken evenly. 1 mL of gas was extracted from each anaerobic tube by a microsyringe and injected into a gas chromatograph to determine the amount of ethylene produced.

TABLE 5
Amount of product ethylene produced in determination
experiment of nitrogenase activity

| Treatment of enzyme | Peak area of |
|---|---|
| activity experiment | product ethylene |
| Without addition of bacteria | ND |
| With addition of bacteria | 1722315 ± 544794 |

As can be seen from the determination results in Table 5, when the Klebsiella variicola LWNF004 was added into the anaerobic tube, it can catalyze the conversion of acetylene to ethylene, while no ethylene production was detected in the control check without the addition of bacteria. Based on the actual measured concentration/area ratio of acetylene standard gas, the nitrogenase activity of the Klebsiella variicola LWNF004 can be calculated to be 8.8 nmol C₂H₄/h vial. The aforementioned results fully demonstrate that the Klebsiella variicola LWNF004 has relatively high nitrogenase activity.

3. Effect of Klebsiella variicola LWNF004 on Corn Roots.

The Klebsiella variicola LWNF004 was inoculated into an LB medium and cultured at 30° C. and 220 RPM for 16 h. The mixture was centrifuged at 5500 rpm for 5 mint to collect the bacteria, and the supernatant was discarded. The concentration of the bacteria was adjusted to 10⁹ CFU/mL with sterile PBS. Corn seeds were planted in the greenhouse, 1000 μL of the prepared bacterial suspension was added, and subsequently, 10 mL of sterile water was added. In a control check (CK), 1000 μL of sterile PBS and 10 mL of sterile water were added. Each of the two treatments was repeated 4 times. After 7 weeks of planting, the roots were taken, the covering soil was removed, and the fresh weight of the roots was measured.

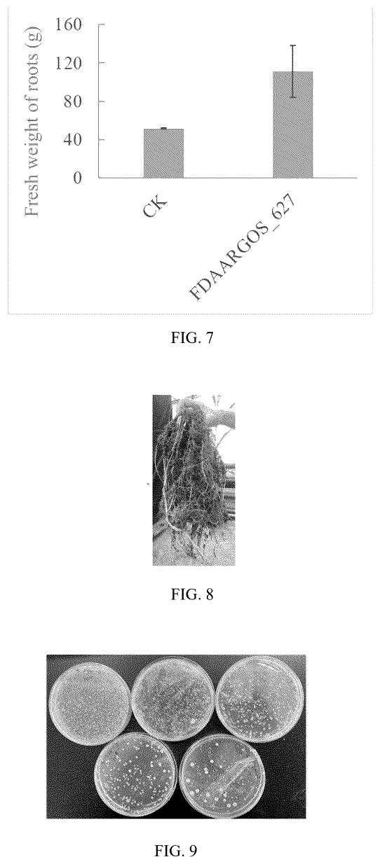

The results are shown in FIG. 7. After 7 weeks of planting, the fresh weight of the corn roots in the control check not treated with the Klebsiella variicola LWNF004 was 52 g, and the fresh weight of the corn roots in the experimental group treated with the Klebsiella variicola LWNF004 was 111 g, which was increased by 116% compared with the control check. The aforementioned results show that the strain can significantly promote the growth of corn roots.

4. Determination of Colonization Abundance of Klebsiella variicola LWNF004.

A gene editing method was used to enable the Klebsiella variicola LWNF004 to carry a red fluorescent protein marker, and subsequently, the marked strain was used for carrying out the corn seedlings experiment. The Klebsiella variicola LWNF004 carrying the fluorescent protein marker was inoculated into an LB medium and cultured at 30° C. and 220 RPM for 16 h. The mixture was centrifuged at 5500 rpm for 5 mint to collect the bacteria, and the supernatant was discarded. The OD value of the bacteria was adjusted to 10⁹ CFU/mL with sterile PBS. Corn seeds were planted in the greenhouse, 1000 μL of the prepared bacterial suspension was added, and subsequently, 10 mL of sterile water was added, with 4 replicates. After nine weeks of growth, root samples were taken (see FIG. 8). 0.25 g of each root sample was collected by mixing lateral roots, root tips, and middle portions of the roots. The collected samples were diluted with a certain ratio of sterile water and subsequently disrupted using a vortex mixer. After that, the original solution was subjected to a serial dilution to 105. The diluted samples were subsequently applied onto LB solid media and cultured under an aerobic condition at 30° C. for 2 days. After culture, the plates were placed at 4° C. for one day to allow the full development of red fluorescence, and subsequently, the bacteria were counted (see FIG. 9).

According to the results of fluorescence counting, the colonization abundance of the Klebsiella variicola in corn roots was still 5×10⁴ CFU/g for each fresh root. The abundance of other Klebsiella nitrogen-fixing bacteria isolated at the same time can only be maintained for 3 weeks.

### 5. Preparation of Microbial Agent

In order to facilitate storage and transportation, the screened Klebsiella variicola LWNF004 was prepared into a lyophilized powder microbial agent. The specific procedures were as follows (the preparation process is the same as FIG. 6):
(1) a fermentation broth of the Klebsiella variicola LWNF004 was centrifuged and separated to obtain a bacterial precipitate;
(2) a lyoprotectant was prepared by mixing skim milk powder, sodium glutamate, glycerol, sucrose, and water in a mass ratio of 75:15:70:10:330;
(3) the lyoprotectant was added to the bacterial precipitate obtained in the aforementioned step (1) in a mass ratio of 1:1, the bacteria and the protectant were mixed fully and equilibrated for 60 min, and subsequently, the mixture was frozen at −20° C. overnight; and
(4) the mixture was lyophilized under an aseptic condition to prepare a lyophilized powder for later use.

An effective viable count of the Klebsiella variicola LWNF004 lyophilized powder is 10¹⁰-10¹¹ CFU/g, and the lyophilized powder can be stored for 6 months at room temperature without reduction of viability.

### Example 3. Effect of Composite Microbial Agent of Klebsiella variicola LWNF004 and Kosakonia sacchari LWNF014 on Corn Growth

The Klebsiella variicola LWNF004 and the Kosakonia sacchari LWNF014 were separately inoculated into an LB medium and cultured at 30° C. and 220 RPM for 16 h. The mixtures were centrifuged at 5500 rpm for 5 mint to collect the bacteria, and the supernatants were discarded. The OD values of the bacteria were adjusted to 2×10⁹ CFU/mL with sterile PBS. Moreover, the Klebsiella variicola LWNF004 was mixed with an equal volume of the Kosakonia sacchari LWNF014 as a bacterial suspension in a mixed microbial agent experimental group. The Klebsiella variicola LWNF004 was mixed with an equal volume of sterile PBS and the Kosakonia sacchari LWNF014 was mixed with an equal volume of sterile PBS as bacterial suspensions in single strain microbial agent control checks, respectively. Sterile PBS was used as a sterile control check. Corn seeds were planted in 50 mL centrifuge tubes. In the experimental group and the single strain microbial agent control checks, 200 μL of the prepared corresponding bacterial suspensions were added, respectively, and subsequently, 10 mL of sterile water was added. In the sterile control check, 200 μL of sterile PBS and 10 mL of sterile water were added. Each treatment was repeated 4 times. After that, 5 mL of water was added every other day. After 2 weeks of planting, the aerial part of the corn was collected and the fresh weight was measured.

The corn growth is shown in Table 6. After 2 weeks of cultivation, the fresh weight of the aerial part of the corn plant in the experimental group applied with the composite microbial agent of the Klebsiella variicola LWNF004 and the Kosakonia sacchari LWNF014 was 1.40 g, the fresh weight of the aerial part of the corn plant in the control check applied with the single strain Klebsiella variicola LWNF004 was 0.93 g, the fresh weight of the aerial part of the corn plant in the control check applied with the single strain Kosakonia sacchari LWNF014 was 1.13 g, and the fresh weight of the aerial part of the corn plant in the sterile control check was 0.67 g. Compared with the sterile control check, the application of the single strain Klebsiella variicola LWNF004, the application of the single strain Kosakonia sacchari LWNF014, and the application of the composite microbial agent of the Klebsiella variicola LWNF004 and the Kosakonia sacchari LWNF014 increased the fresh weight of the aerial parts of the corn by 38.9%, 67.9%, and 109.0%, respectively. The plant height of the composite microbial agent experimental group increased by 50.5% and 23.9%, respectively, compared with those of the two single strain control checks. The aforementioned results fully demonstrate that the composite microbial agent of the Klebsiella variicola LWNF004 and the Kosakonia sacchari LWNF014 can further promote corn growth.

TABLE 6
Effect of composite microbial agent of Klebsiella variicola
LWNF004 and Kosakonia sacchari LWNF014 on
fresh weight of corn plants

| Strain | Fresh weight of aerial part (g) |
|---|---|
| Sterile control | 0.67 |
| Klebsiella variicola LWNF004 | 0.93 |
| Kosakonia sacchari LWNF014 | 1.13 |
| Composite microbial agent | 1.40 |
| of Klebsiella variicola | |
| LWNF004 and Kosakonia | |
| sacchari LWNF014 | |

Although the present invention has been described in detail with respect to the general description and the specific embodiments thereof, it will be apparent to those skilled in the art that modifications or improvements can be made based on the present invention. Accordingly, these modifications or improvements made without departing from the spirit of the present invention fall within the scope of protection of the present invention.

1.-10. (canceled)

11. A strain of Kosakonia sacchari LWNF014, wherein the strain is classified and named as Kosakonia sacchari with an accession number of CGMCC No. 28352.

12. A strain of Klebsiella variicola LWNF004, wherein the strain is classified and named as Klebsiella variicola with an accession number of CGMCC No. 28351.

13. A method for promoting plant growth, wherein the method comprises using the Kosakonia sacchari LWNF014 according to claim 11 and/or the Klebsiella variicola LWNF004 according to claim 12.

14. The method according to claim 13, wherein the plant is a crop comprising a food crop and a cash crop.

15. The method according to claim 14, wherein the cash crop comprises an oil crop and a vegetable crop; the food crop comprises a grain crop, a tuber crop, and an edible legume crop.

16. The method according to claim 15, wherein the crop is a vegetable crop comprising corn, beans, cucumbers or peppers.

17. The method according to any one of claims 13-16, wherein the method can increase the nitrogen content of the plant, promote photosynthesis, and increase the chlorophyll content, or promote a growth-promoting effect for plant growth.

18. A microbial agent, comprising the Kosakonia sacchari LWNF014 according to claim 11 and/or the Klebsiella variicola LWNF004 according to claim 12.

19. A method for preparing the microbial agent according to claim 18, wherein the Kosakonia sacchari LWNF014 according to claim 11 and/or the Klebsiella variicola LWNF004 according to claim 12 are activated to prepare a seed solution, which is subsequently inoculated into a fermentation medium for expansion to a stationary phase, and the microbial agent is obtained after separation.

20. A method for preparing the microbial agent according to claim 18, wherein the microbial agent is prepared into a lyophilized powder microbial fertilizer; specifically, a fermentation broth obtained by fermentation of the Kosakonia sacchari LWNF014 according to claim 11 and/or the Klebsiella variicola LWNF004 according to claim 12 is centrifuged and separated to obtain a bacterial precipitate, a lyoprotectant is subsequently added, and the mixture is lyophilized under an aseptic condition to prepare a lyophilized powder.

21. A plant photosynthesis improver, comprising the Kosakonia sacchari LWNF014 according to claim 11 and/or the Klebsiella variicola LWNF004 according to claim 12, or the microbial agent according to claim 18, or the microbial agent prepared by the method according to any one of claims 19-20.

22. A plant nitrogen nutrition improver, comprising the Kosakonia sacchari LWNF014 according to claim 11 and/or the Klebsiella variicola LWNF004 according to claim 12, or the microbial agent according to claim 18, or the microbial agent prepared by the method according to any one of claims 19-20.

23. A plant growth and development promoter, comprising the Kosakonia sacchari LWNF014 according to claim 11 and/or the Klebsiella variicola LWNF004 according to claim 12, or the microbial agent according to claim 18, or the microbial agent prepared by the method according to any one of claims 19-20.
